(19) United States
(12) Patent Application Publication
(10) Pub. No.: US 2012/0100249 A1
(43) Pub. Date: Apr. 26, 2012

(54) NOVEL ASPARAGINASE ENZYME

(75) Inventors: JAN METSKE VAN DER LAAN, BREDA (NL); IISE DE LANGE, HELLEVOETSLUIS (NL); MARK CRISTIAAN STOR, GOUDA (NL)

(73) Assignee: DSM IP ASSETS B.V., HEERLEN (NL)

(21) Appl. No.: 13/092,466

(22) Filed: Apr. 22, 2011

(30) Foreign Application Priority Data
Apr. 27, 2010 (EP) 10161214.1

(51) Int. Cl. C12N 9/78; A23L 1/305; C12N 1/21; C12N 1/15; C12N 15/55; C12N 15/63

(52) U.S. Cl. 426/7; 435/227; 536/23.2; 435/320.1; 435/252.3; 435/254.11; 426/61

(57) ABSTRACT

The present invention relates to a protein which exhibits asparaginase activity and which has an amino acid sequence according to SEQ ID NO. 2-SEQ ID NO.10. The advantage of the protein of the present invention is that it exhibits asparaginase activity (EC 3.5.1.1) with a specific activity at acidic, neutral and alkaline pH which is many times higher than the specific activity of wild-type asparaginase.

### FIELD OF THE INVENTION

The present invention relates to a novel protein and to polynucleotide sequences encoding the protein. More particular, it relates to a protein with asparaginase activity and to methods of using these proteins.

### BACKGROUND OF THE INVENTION

Recently, the occurrence of acrylamide in a number of heated food products was published (Tareke et al. Chem. Res. Toxicol. 13, 517-522 (2000)). Since acrylamide is considered as probably carcinogenic for animals and humans, this finding had resulted in world-wide concern. Further research revealed that considerable amounts of acrylamide are detectable in a variety of baked, fried and oven prepared common foods and it was demonstrated that the occurrence of acrylamide in food was the result of the heating process. A pathway for the formation of acrylamide from amino acids and reducing sugars as a result of the Maillard reaction has been proposed by Mottram et al. Nature 419:448 (2002). According to this hypothesis, acrylamide may be formed during the Maillard reaction. During baking and roasting, the Maillard reaction is mainly responsible for the color, smell and taste. A reaction associated with the Maillard is the Strecker degradation of amino acids and a pathway to acrylamide was proposed. The formation of acrylamide became detectable when the temperature exceeded 120° C., and the highest formation rate was observed at around 170° C. When asparagine and glucose were present, the highest levels of acrylamide could be observed, while glutamine and aspartic acid only resulted in trace quantities.

The official migration limit in the EU for acrylamide migrating into food from food contact plastics is set at 10 ppb (10 micrograms per kilogram). Although no official limit is yet set for acrylamide that forms during cooking, the fact that a lot of products exceed this value, especially cereals, bread products and potato or corn based products, causes concern.

Several plant raw materials are known to contain substantial levels of asparagine. In potatoes asparagine is the dominant free amino acid (940 mg/kg, corresponding with 40% of the total amino-acid content) and in wheat flour asparagine is present as a level of about 167 mg/kg, corresponding with 14% of the total free amino acids pool (Belitz and Grosch in Food Chemistry—Springer New York, 1999). The fact that acrylamide is formed mainly from asparagine (combined with reducing sugars) may explain the high levels of acrylamide in fried, oven-cooked or roasted plant products. Therefore, in the interest of public health, there is an urgent need for food products that have substantially lower levels of acrylamide or, preferably, are devoid of it.

A variety of solutions to decrease the acrylamide content has been proposed, either by altering processing variables, e.g. temperature or duration of the heating step, or by chemically or enzymatically preventing the formation of acrylamide or by removing formed acrylamide.

In several patent applications the use of asparaginase for decreasing the level of asparagine and thereby the amount of acrylamide formed has been disclosed. Suitable asparaginases for this purpose have been yielded from several fungal sources, as for example Aspergillus niger in WO2004/030468 and Aspergillus oryzae in WO04/032648.

Although all L-asparaginases catalyze the same chemical conversion, this does not mean that they are suitable for the same applications. Various applications will place different demands on the conditions under which the enzymes have to operate. Physical and chemical parameters that may influence the rate of an enzymatic conversion are the temperature (which has a positive effect on the chemical reaction rates, but may have a negative effect on enzyme stability), the moisture content, the pH, the salt concentration, the structural integrity of the food matrix, the presence of activators or inhibitors of the enzyme, the concentration of the substrate and products, etc. Therefore there exists an ongoing need for improved asparaginases for several applications having improved properties.

### DETAILED DESCRIPTION OF THE INVENTION

The present invention relates to a polypeptide which exhibits asparaginase activity and which shows a specific activity which is (i) at least twice the specific activity of A. niger wild type asparaginase of SEQ ID NO: 1 at a pH between pH 5 and pH 8, at a temperature of 37° C., and (ii) which is at least 10% higher at pH 5, pH 6 and pH 7 than the specific activity of variant asparaginase ASN02 from WO 2008/128974 (depicted in SEQ ID NO: 20 of the present patent application and corresponding to SEQ ID NO: 5 in WO2008/128974) at these pH values. Such proteins have an aspartic acid, a glycine or histidine at position 63 and a serine, phenylalanine, or valine at position 88 of SEQ ID NO.1, which is the sequence of A. niger wild type asparaginase as published in WO 04/030468. Preferably this polypeptide has a degree of identity (% identity) of at least 90%, preferably at least 95% to the wild type A. niger asparaginase depicted in SEQ ID NO: 1.

The terms “homology” or “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Preferably, the two sequences are the same length.

A sequence comparison may be carried out over the entire lengths of the two sequences being compared or over fragment of the two sequences. Typically, the comparison will be carried out over the full length of the two sequences being compared. However, sequence identity may be carried out over a region of, for example, twenty, fifty, one hundred or more contiguous amino acid residues.

The skilled person will be aware of the fact that several different computer programs are available to determine the homology between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

The nucleotide or the protein sequences of the present invention can further be used as a “query sequence” to perform a search against public databases to, for example, identify other family members or related sequences. Such searches can be performed using the BLASTN and BLASTP programs (version 2.0) of Altschul, et al. (1990) J. Mol. Biol. 215:403-10. BLAST protein searches can be performed with the BLASTP program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTP and BLASTN) can be used. See the homepage of the National Center for Biotechnology Information at http://www.ncbi.nlm.nih.gov/.

One advantage of the proteins of the present invention is that they exhibit asparaginase activity (EC 3.5.1.1) with high specific activity at neutral, acidic and alkaline pH. In some embodiments, the specific activity is more than 6 times (pH 7) or more than 30 times (pH 8) as high as that of wild-type asparaginase at these pH values.

In the present application, the term ‘specific activity’ refers to the asparaginase activity measured in units/mg of asparaginase protein.

The protein according to the invention may be obtained in any suitable way. In one embodiment, the protein is obtained by modifying an asparaginase. A suitable asparaginase for modification may be obtained from various sources, such as for example from a plant, an animal or a microorganism. For example, an asparaginase may be obtained from Escherichia, Erwinia, Streptomyces, Pseudomonas, Aspergillus and Bacillus species. An example of a suitable Escherichia strain is Escherichia coli. An example of a suitable Erwinia strain is Erwinia chrysanthemi. Examples of suitable Streptomyces strains are Streptomyces lividans and Streptomyces murinus. Examples of suitable Aspergillus strains are Aspergillus oryzae, Aspergillus nidulans or Aspergillus niger. Examples of suitable Bacillus strains are Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis or Bacillus thurigiensis.

An example of methods suitable for obtaining asparaginase from Bacillus, Streptomyces, Escherichia or Pseudomonas strains is described in WO 03/083043. An example of methods suitable for obtaining asparaginase from Aspergillus is described in WO 2004/030468 and WO 04/032648.

A preferred asparaginase for modification to obtain the protein according the invention is the asparaginase having the sequence set out in SEQ ID NO: 1.

In one embodiment, a protein S1 according to the invention is having an amino acid sequence according to SEQ ID NO. 2, or part of that sequence covering amino acids 16-378, 17-378, 18-378, 19-378, 20-378, 21-378, 22-378, 23-378, 24-378, 25-378 or 26-378. These various forms may arise due to processing of the signal sequence, and possible further truncation, depending on the specific host used and culturing conditions. In this embodiment, compared to SEQ ID NO. 1, Ser at position 16 is replaced by Ala, Asp at position 63 is replaced by Gly, Gly at position 132 is replaced by Ser and Ala at position 293 is replaced by Val.

In another embodiment, a protein S2 according to the invention is having an amino acid sequence according to SEQ ID NO. 3, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Thr at position 41 is replaced by Ile, Asp at position 63 is replaced by Gly, Ser at position 88 is replaced by Val, Asp at position 111 is replaced by Gly and Arg at position 122 is replaced by His.

In another embodiment, a protein S3 according to the invention is having an amino acid sequence according to SEQ ID NO.4, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO.1, Thr at position 41 is replaced by Ile, Asp at position 63 is replaced by Gly and Ser at position 88 is replaced by Phe.

In another embodiment, protein S4 according to the invention is having an amino acid sequence according to SEQ ID NO.5, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Asp at position 63 is replaced by His, Ala at position 76 is replaced by Thr, Val at position 77 is replaced by Phe, Ala at position 101 is replaced by Val and Ala at position 170 is replaced by Thr.

In another embodiment, a protein S5 according to the invention is having an amino acid sequence according to SEQ ID NO.6, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Ala at position 17 is replaced by Thr, Asp at position 63 is replaced by Gly, Lys at position 119 is replaced by Asn, Arg at position 262 is replaced by Cys.

In another embodiment, a protein S6 according to the invention is having an amino acid sequence according to SEQ ID NO.7, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Thr at position 41 is replaced by Ile, Ser at position 66 is replaced by Pro, Ser at position 88 is replaced by Val, Val at position 244 is replaced by Ala and Arg at position 262 is replaced by Cys.

In another embodiment, a protein S7 according to the invention is having an amino acid sequence according to SEQ ID NO.8, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Asp at position 63 is replaced by His, Ala at position 76 is replaced by Thr, Val at position 77 is replaced by Phe, Ala at position 101 is replaced by Val, Asp at position 111 is replaced by Gly, Ile at position 161 is replaced by Leu, Ala at position 170 is replaced by Thr, Val at position 244 is replaced by Ala and Val at position 371 is replaced by Met.

In another embodiment, a protein S8 according to the invention is having an amino acid sequence according to SEQ ID NO.9, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Ser at position 16 is replaced by Ala, Asp at position 63 is replaced by Gly, Ala at position 76 is replaced by Thr and Lys at position 119 is replaced by Asn.

In another embodiment, a protein S9 according to the invention is having an amino acid sequence according to SEQ ID NO.10, or part of that sequence covering amino acids 16-378, 17-378, 18-378, 19-378, 20-378, 21-378, 22-378, 23-378, 24-378, 25-378 or 26-378. In this embodiment, in comparison to SEQ ID NO. 1, Glu at position 28 is replaced by Gly, Thr at position 33 is replaced by Ala and Asp at position 63 is replaced by His.

In another embodiment, the protein according to the invention is obtained by expression of a nucleotide sequence which encodes the amino acid sequence of the protein according to the invention. Therefore, in another aspect, the present invention relates to a nucleic acid molecule with a nucleotide sequence which encodes a polypeptide according to the invention. One example of a nucleotide sequence which encodes the protein according the invention is given in SEQ ID NO. 11 (encoding S1). Other DNA sequences encoding a protein according to the invention are given in SEQ ID NO. 12 (encoding S2), SEQ ID NO. 13 (encoding S3), SEQ ID NO. 14 (encoding S4), SEQ ID NO. 15 (encoding S5), SEQ ID NO. 16 (encoding S6), SEQ ID NO. 17 (encoding S7), SEQ ID NO. 18 (encoding S8) and SEQ ID NO. 19 (encoding S9).

Alternatively, degenerate nucleotide sequences may be used. Methods for synthesizing degenerate nucleotide sequences are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

A nucleic acid molecule according to the present invention may be generated using standard molecular biology techniques well known to those skilled in the art. For example, using standard synthetic techniques, the required nucleic acid molecule may be synthesized de novo. Such a synthetic process will typically be an automated process.

Alternatively, a nucleic acid molecule of the invention may be generated by using other methods well known to those skilled in the art.

A nucleic acid molecule derived in this way can be cloned into an appropriate vector and characterized by DNA sequence analysis.

Therefore, in another aspect, the present invention relates to a nucleotide construct comprising a nucleic acid molecule according to the invention. In one embodiment, the nucleotide construct according to the invention is a vector, such as a cloning vector or expression vector. The vector may be prokaryotic or eukaryotic, but is preferably a eukaryotic vector. In another embodiment, the nucleotide construct according to the invention is a plasmid. For example, a plasmid for autonomous replication in a prokaryotic or eukaryotic host.

The vector or plasmid typically comprises one or more regulatory sequences operatively linked to the nucleic acid sequence to be expressed to allow expression of the sequence in the specific host. Such regulatory sequences include promoters, enhancers and other expression control elements and are known in the art. See for example Goeddel; Gene Expression Technology: Methods in Enzymology 185, Academic Press, San Diego, Calif. (1990). It will be appreciated by those skilled in the art that the design of the expression vector depends on such factors as the choice of the host cell to be transformed and the level of expression of protein desired. Suitable promoters are known to the skilled person. In a specific embodiment, promoters are preferred that are capable of directing a high expression level of asparaginase in filamentous fungi. Such promoters are known in the art. The expression constructs may contain sites for transcription initiation, termination and a ribosome binding site for translation. The coding portion of the mature transcripts expressed by the constructs will include a translation initiating AUG at the beginning and a termination codon appropriately positioned at the end of the polypeptide to be translated. For secretion of the translated protein into the lumen of the endoplasmic reticulum, into the periplasmic space or into the extracellular environment, appropriate secretion signal may be incorporated into the coding sequence of the encoded protein.

The vector or plasmid typically also contains one or more selectable markers. Preferred selectable markers include those which confer resistance to drugs, such as G418, hygromycin and methatrexate. Nucleic acid encoding a selectable marker can be introduced into a host cell on the same vector as that encoding the protein according to the invention or can be introduced on a separate vector. Cells stably transfected with the introduced nucleic acid can be identified by drug selection.

Expression vectors useful in the present invention include chromosomal-, episomal- and virus-derived vectors, such as vectors derived from bacterial plasmids, bacteriophage, yeast episome, yeast chromosomal elements, viruses such as baculoviruses, papova viruses, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids.

Vector DNA can be introduced into prokaryotic or eukaryotic cells via conventional transformation or transfection techniques. As used herein, the terms “transformation” and “transfection” are intended to refer to a variety of art—recognized techniques for introducing foreign nucleic acid (e.g., DNA) into a host cell. For suitable methods for transforming or transfecting host cells see Sambrook, et al. (Molecular Cloning: A Laboratory Manual, 2nd, ed. Cold Spring Harbor Laboratory, Cold Spring Harbor Laboratory Press, Cold Spring Harbor, N.Y., 1989), Davis et al., Basic Methods in Molecular Biology (1986) and other laboratory manuals.

Also nucleic acid molecule which are antisense to a nucleic acid molecule according to the invention are encompassed by the present invention.

In another aspect, the present invention relates to a cell which is transformed with a nucleotide construct according to the invention which thus acts as a host cell. Both prokaryotic and eukaryotic cells, such as bacteria, fungi, yeast, plant and mammalian cells, may acts as a host cell. Suitable example include bacterial cells, such as E. coli, Streptomyces, Salmonella typhimurium and certain Bacillus species; fungal cells such as Aspergillus species, for example A. niger, A. oryzae and A. nidulans, such as yeast such as Kluyveromyces, for example K. lactis and/or Puchia, for example P. pastoris; insect cells such as Drosophila S2 and Spodoptera Sf9; animal cells such as CHO, VERO, BHK, HeLa, 3T3 and COS; and plant cells. Especially preferred are cells from filamentous fungi, in particular Aspergillus niger. Appropriate culture mediums and conditions for the above-described host cells are known in the art.

A host cell can be chosen that modulates the expression of the inserted sequences, or modifies and processes the product encoded by the incorporated nucleic acid sequence in a specific, desired fashion. Such modifications (e.g., glycosylation) and processing (e.g., cleavage) of protein products may facilitate optimal functioning of the encoded protein.

In another aspect, the present invention relates to the use of a protein according to the invention in the food industry. In one embodiment, the protein of the invention is conveniently used to prevent or diminish the formation of acrylamide in food products, especially in a thermally processed food product based on an asparagine-containing raw material. For example, the protein may be used in a process for the production of a food product involving at least one heating step, comprising adding one or more asparaginase enzymes to an intermediate form of the food product, whereby the enzyme is added prior to the heating step in an amount that is effective in reducing the level of asparaginase that is present in the intermediate form of the food product. Such a process is disclosed in WO04/030468 which process and all its preferences are herein incorporated by reference. Also in WO04/026043 suitable processes are described wherein the protein according to the invention could be used. The processes disclosed in WO 04/026043 and all preferences disclosed are herein incorporated by reference.

An intermediate form of the food product is defined herein as any form that occurs during the production process prior to obtaining the final form of the food product. The intermediate form may comprise the individual raw materials used and/or mixture thereof and/or mixtures with additives and/or processing aids, or subsequently processed form thereof. For example, for the food product bread, the intermediate forms comprise for example wheat, wheat flour, the initial mixture thereof with other bread ingredients such as for example water, salt, yeast and bread improving compositions, the mixed dough, the kneaded dough, the leavened dough and the partially baked dough. For example for several potato based products, dehydrated potato flakes or granules are intermediate products, and corn mass is an intermediate product for tortilla chips.

The food product may be made from at least one raw material that is of plant origin, for example potato, tobacco, coffee, cocoa, rice, cereal, for example wheat, rye corn, maize, barley, groats, buckwheat and oat. In the present context, the term ‘wheat’ encompasses all known species of the Triticum genus, for example aestivum, durum and/or spelta. Also food products made from more than one raw material or intermediate are included in the scope of this invention, for example food products comprising both wheat (flour and/or starch) and potato. Examples of food products to which the process according the invention can suitably be applied are any flour based products—for example bread, baguettes, doughnuts, rolls, crackers, pastry, cake, pretzels, bagels, Dutch honey cake, cookies, biscuits, gingerbread, gingercake and crispbread —, and any potato-based products—for example French fries, pommes frites, potato chips, potato crisps, croquettes, fabricated potato snacks, or corn-based product—for example corn chips or tortilla chips.

Raw materials as cited above are known to contain substantial amounts of asparagine which is involved in the formation of acrylamide during the heating step of the production process. Alternatively, the asparagine may originate from other sources than the raw materials e.g. from protein hydrolysates, such as yeast extracts, soy hydrolysate, casein hydrolysate and the like, which are used as an additive in the food production process. A preferred production process is the baking of bread and other baked products from wheat flour and/or flours from other cereal origin. Another preferred production process is the deep-frying of potato chips from potato slices.

Preferred heating steps are those at which at least a part of the intermediate food product, e.g. the surface of the food product, is exposed to temperatures at which the formation of acrylamide is promoted, e.g. 110° C. or higher, 120° C. or higher temperatures. The heating step in the process according to the invention may be carried out in ovens, for instance at a temperature between 180-220° C., such as for the baking of bread and other bakery products, or in oil such as the frying of potato chips, for example at 160-190° C.

An additional application for the protein according to the invention is its use in the therapy of tumours. The metabolism of tumour cells requires L-asparagine, which can quickly be degraded by asparaginases. The protein according to the invention may also be used as an adjunct in treatment of some human leukaemia. Administration of asparaginase in experimental animals and humans leads to regression of certain lymphomas and leukemia. Therefore in one embodiment the invention relates to the use of the protein according to the invention for use as medicament, e.g. in the treatment of tumors, e.g. in the treatment of lymphomas or leukaemia in animals or humans.

In all the above-mentioned applications, the protein may be used as such or it may be used in a composition. Therefore, in another aspect, the present invention relates to a composition comprising a protein or a nucleotide sequence according to the invention. The composition according to the invention may comprise other ingredients, such as further enzymes, such as lipolytic enzymes (such as phospholipase, galactolipase, triacyl glycerol lipase), esterases, cellulases, hemicellulase (such as xylanase) amylases (such as α-amylase, β-amylase, maltogenic amylase), proteases; nucleotides, excipients, fillers or adjuvants.

### Example 1

### Production and Purification of Asparaginases According to the Invention

Asparaginase S7 of the invention, having an amino acid sequence as depicted in SEQ ID NO.8, was obtained by the construction of an expression plasmid containing a DNA sequence as depicted in SEQ ID No. 17., transforming an Aspergillus niger strain with the plasmid and growing the Aspergillus niger strains as described in WO 2004/030468.

After growing Aspergillus niger containing the proper expression plasmid, cell free supernatant was prepared by centrifugation of the fermentation broth at 5000×g for 30 minutes at 4° C. The supernatants was filtered further over a Miracloth filter (Calbiochem cat #475855) and a GF/A Whatmann Glass microfiber filter (150 mm \acute{Ø}), respectively, to remove any solids. To remove any fungal material the supernatant was adjusted to pH 5 with 4N KOH and sterile filtrated over a 2 μm (bottle-top) filter with suction. The supernatant was stored until use at 4° C. or frozen at −20° C.

The asparaginase was purified by anion ion-exchange chromatography starting from cell free supernatant and ccUF desalted via a PD-10 column (Amersham Biosciences). The desalted material was applied to a Mono-Q or Q-Sepharose column equilibrated in 20 mM histidine buffer pH 5.96. After extensive washing the asparaginase was eluted from the column using a gradient from 0 to 1M NaCl.

Other variants according to the invention, S1, S2, S3, S4, S5, S6, S8 and S9, with amino acid sequences as depicted in SEQ ID No. 2, SEQ ID No. 3, SEQ ID No. 4, SEQ ID No. 5, SEQ ID No. 6, SEQ ID No. 7, SEQ ID No. 9 and SEQ ID NO. 10 can be produced and isolated in the same way using the DNA sequences as depicted in SEQ ID No. 11, SEQ ID No. 12, SEQ ID No. 13, SEQ ID No. 14, SEQ ID No. 15, SEQ ID No. 16, SEQ ID No. 18 and SEQ ID No. 19, respectively.

### Example 2

### Specific Activity as a Function of pH

The specific activity of the asparaginase variants was determined at pH 4, pH 5, pH 6, pH 7, pH 8 at 37° C. in 50 mM phosphate/citrate buffer using cell-free supernatants. The specific activity is measured by dividing the activity of a sample (in units/ml) by mg protein/ml asparaginase present in the sample.

The asparaginase activity was measured using L-asparagine as substrate. The amount of ammonia that was liberated by the action of the enzyme was measured according to the Berthelot reaction. Ready-to-use reagents phenolnitroprusside and alkaline hypoclorite were obtained from Sigma. 100 μl enzyme sample was mixed with 2000 μl 100 mM L-asparagine in a mixture of 50 mM citric acid and 50 mM sodium pyrophosphate buffer of the desired pH. After incubation at 37° C. for 30 minutes the reaction was stopped by adding 400 μl 25% trichloroacetic acid, whereafter 2500 μl water was added. During the incubation the temperature was fixed at 37° C. unless indicated otherwise.

It should be understood by a person skilled in the art that enzyme dosing was chosen in such a way that after incubation under the above conditions a signal was obtained significantly above the background but still within a range where the signals obtained are proportional to the amount of enzyme added. Preferably the reaction was zero order.

After stopping the reaction, 4 μl of the incubation mixture was added to 156 μl water. Subsequently 34 μl phenol/nitroprusside solution (Sigma P6994) and 34 μl alkaline hypochlorite solution (Sigma A1727) were added. After 676 seconds of incubation at 37° C., the extinction was measured at 600 nm. Readings were corrected for the background signal by including the appropriate blanks. A sample with (TCA) inactivated enzyme was used as a blank. The assays were run on an autoanalyzer e.g. a Konelab Arena 30 (Thermo Scientific). The activity was determined using a calibration line made up by plotting the measured absorbance at 600 nm versus the known ammonium sulphate concentrations of a standard series. Activity was expressed in units, where one unit is defined as the amount of enzyme required to liberate one micromole of ammonia from L-asparagine per minute under defined assay conditions.

The amount of asparaginase protein in the cell-free supernatants was determined by PAA-SDS gel electrophoresis using NuPAGE® Novex 4-12% Bis-Tris 12 well gels (Invitrogen, NP0322BOX). 1 μl of culture supernatant was incubated with 1 μl 10×NuPAGE® Sample Reducing Agent (Invitrogen, NP0004), 2.5 μl 4×NuPAGE LDS Sample Buffer (Invitrogen, NP0007) and 5.5 μl milliQ water for 10 minutes at 70° C. The resulting reduced sample was loaded on the gel. The SeeBlue® Plus2 prestained standard (Invitrogen, LC5925) was used as size marker. In addition, 0.5 μg of BSA (Sigma A9418) was loaded as calibrator for the amount of protein. The gels were run in NuPAGE® MES SDS running buffer (Invitrogen, NP0002), containing NuPAGE® Antioxidant (Invitrogen, NP0005) for 35 minutes at 200 V. Following electrophoresis, the gels were fixed for 2×30 minutes in Fix solution (7% HAc (v/v) and 10% ethanol (v/v)), stained over night with SYPRO Ruby protein gel stain (Invitrogen S12000) and de-stained in Fix solution for 2×30 minutes. Subsequently, the gels were washed with demineralised water and scanned with the Typhoon 9200 scanner (GE Healthcare). The peak volume was calculated using Image Quant TLv2003.02 software and the protein concentrations were calculated based on the BSA protein band.

The improvement factors for the specific activity of the asparaginases according to the invention compared to ASN02 (WO2008/128974) (SEQ ID NO. 20 in the present patent application) and to Aspergillus niger wild type asparaginase (SEQ ID NO. 1 in the present patent application) at different pH values are shown in Table 1.

TABLE 1

| | Mutant | pH 4 | pH 5 | pH 6 | pH 7 | pH 8 |
|---|---|---|---|---|---|---|
| WT | | 65% | 57% | 35% | 20% | 9% |
| ASN02 | D63G + D111G + | 100% | 100% | 100% | 100% | 100% |
| | R122H | | | | | |
| S1 | S16A + D63G + | 74% | 138% | 123% | 154% | 97% |
| | G132S + A293V | | | | | |
| S2 | T41I + D63G + | 162% | 167% | 182% | 198% | 319% |
| | S88V + D111G + | | | | | |
| | R122H | | | | | |
| S3 | T41I + D63G + | 177% | 184% | 192% | 200% | 296% |
| | S88F | | | | | |
| S5 | A17T + D63G + | 149% | 157% | 167% | 169% | 59% |
| | K119N + R262C | | | | | |
| S6 | T41I + S66P + | 128% | 161% | 133% | 134% | 32% |
| | S88V + V244A + | | | | | |
| | R262C | | | | | |

The results show that the activity of the variants increased over the whole pH range in comparison to the activity of wild type asparaginase. In addition, the activity profile of the variants has shifted in such a way that the enzymes can be applied under more alkaline conditions.

All variants show at least an improvement factor of 10% at neutral and acidic pH compared to the control ASN02.

### Example 3

### Asparaginase Activity at Alkaline pH

Since it was observed that the specific activity profile of the variant according to the invention had shifted towards more alkaline conditions, the pH at which 50% of the activity was retained and the ratio between the asparaginase activity at pH 8 and the asparaginase activity at pH 6—was also determined and is given in Table 2. Aspergillus niger asparaginase (WO2004/030468), ASN02 (WO2008/128974) were used as controls.

Assay to determine ratio between the asparaginase activity at pH 8 and the asparaginase activity at pH 6 was performed in microtiterplates (MTP's) or tubes. Activity was measured at pH 6 and pH 8. 10 μl enzyme sample was mixed with 190 μl 100 mM L-asparagine in 100 mM phosphate buffer pH=6.0 or 100 mM phosphate buffer pH=8.0. After incubation at room temperature and for 1 hr the reaction was stopped by adding 100 μl 12.5% trichloroacetic acid. The enzyme dosing was chosen in such a way that after 1 hour incubation at room temperature, a signal was obtained significantly above the background. After stopping the reaction, 95 μl water was added to 8 μl of the incubation mixture. Subsequently, 70 μl phenol/nitroprusside solution (Sigma P6994) and 70 μl alkaline hypochlorite solution (Sigma A1727) were added. After 60 minutes of incubation at room temperature, the extinction was measured at 620 nm. Readings were corrected for the background signal by including the appropriate blanks e.g. inactivated sample and/or supernatant from fermentation samples of empty host strains. Empty strain indicates a host strain which has not been transformed to contain the asparaginase gene. The activity was determined using a calibration line made up by plotting the measured absorbance at 620 nm versus the known ammonium sulphate concentrations of a standard series. Activity is expressed in units, where one unit is defined as the amount of enzyme required to liberate one micromole of ammonia from L-asparagine per minute under defined assay conditions.

TABLE 2

| | | pH at | Ratio between |
|---|---|---|---|
| | Amino acid substitution if | which still | activity at pH = 8 |
| | compared with wild type | 50% activity | and the activity |
| clone | sequence (WO2004/030468) | is observed | at pH = 6 |
| WT | Aspergillus niger | 6.7 | 0.12 |
| ASN02 | D63G + D111G + R122H | 7.9 | 0.47 |
| S4 | D63H + A76T + V77F + | 8.5 | 1.22 |
| | A101V + A170T | | |
| S7 | D63H + A76T + V77F + | 8.4 | 0.91 |
| | A101V + D111G + I161L + | | |
| | A170T + V244A + V371M | | |
| S8 | S16A + D63G + A76T + | 8.3 | 0.87 |
| | K119N | | |
| S2 | T41I + D63G + S88V + | 8.3 | 0.81 |
| | D111G + R122H | | |
| S9 | E28G + T33A + D63H | 8.3 | 0.79 |
| S3 | T41I + D63G + S88F | 8.3 | 0.72 |

The results emphasize the pH shift to more alkaline pH, since the pH at which the variants still exhibit 50% of their maximal catalytic activity is an indication for how the alkaline limb of the pH activity relationship has shifted towards alkaline pH. Furthermore, the increased ratio between the activity at pH 8 and pH 6 indicate that the new variants have an increased activity at alkaline pH.

1. A polypeptide with an amino acid sequence according to SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO:9 or SEQ ID No.10, or part of that sequence covering amino acids 16-378, 17-378, 18-378, 19-378, 20-378, 21-378, 22-378, 23-378, 24-378, 25-378 or 26-378 and which exhibits asparaginase activity.

2. A polypeptide according to claim 1 which exhibits asparaginase activity and which shows a specific activity which is (i) at least twice the specific activity of A. niger wild type asparaginase of SEQ ID NO: 1 at a pH between pH 5 and pH 8, at 37 degrees C. and (ii) which is at least 10% higher at pH 5, pH 6 and pH 7, at 37 degrees C. than the specific activity of variant asparaginase ASN02 from WO 2008/128974 at these pH values and temperature, wherein the polypeptide has an aspartic acid, a glycine or histidine at position 63 and a serine, a phenylalanine or a valine at position 88 of SEQ ID NO.1.

3. A nucleotide sequence encoding a polypeptide according to claim 1.

4. A nucleotide construct comprising a nucleotide sequence according to claim 3.

5. A nucleotide construct according to claim 4, wherein the construct is a vector.

6. A cell which is transformed with a nucleotide construct according to claim 4.

7. A cell according to claim 6, wherein the cell is a microorganism, preferably a fungus or a bacterium.

8. A process wherein a polypeptide according to claim 1 is used in the food industry.

9. A process according to claim 8, wherein the polypeptide is used to prevent or diminish the formation of acrylamide in food products.

10. A composition comprising a polypeptide according to claim 1.

11. A composition comprising a nucleotide sequence according to claim 3.

12. A composition according to claim 10, which further comprises other enzymes.


SEQUENCE LISTING (20 sequences)

SEQ ID NO: 1 - protein, 378 aa, Aspergillus niger
MPLKPILLSALASLASASPLLYSRTTNETFVFTNANGLNFTQMNTTLPNVTIFATGGTIAGSDSSSTATTGYTSGAVGVLSLIDAVPSMLDVANVAGVQVANVGSEDITSDILISMSKKLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 2 - protein, 378 aa, Artificial sequence
  mutated from Aspergillus niger
MPLKPILLSALASLAAASPLLYSRTTNETFVFTNANGLNFTQMNTTLPNVTIFATGGTIAGSGSSSTATTGYTSGAVGVLSLIDAVPSMLDVANVAGVQVANVGSEDITSDILISMSKKLNRVVCEDPTMASAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGVGAGGVTTSFNEAIEDVINRLEIPVVQSMRTVNGEVPLSDVSSDTATHIASGYLNPQKSRILLGLLLSQGKNITEIADVFALGTDA

SEQ ID NO: 3 - protein, 378 aa, Artificial sequence
  Mutated from Aspergillus niger
MPLKPILLSALASLASASPLLYSRTTNETFVFTNANGLNFIQMNTTLPNVTIFATGGTIAGSGSSSTATTGYTSGAVGVLSLIDAVPVMLDVANVAGVQVANVGSEDITSGILISMSKKLNHVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 4 - protein, 378 aa, Artificial sequence
  Mutated from Aspergillus niger
MPLKPILLSALASLASASPLLYSRTTNETFVFTNANGLNFIQMNTTLPNVTIFATGGTIAGSGSSSTATTGYTSGAVGVLSLIDAVPFMLDVANVAGVQVANVGSEDITSDILISMSKKLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 5 - protein, 378 aa, Artificial sequence
  Mutated from Aspergillus niger
MPLKPILLSALASLASASPLLYSRTTNETFVFTNANGLNFTQMNTTLPNVTIFATGGTIAGSHSSSTATTGYTSGTFGVLSLIDAVPSMLDVANVAGVQVVNVGSEDITSDILISMSKKLNRVVCEDPTMAGAVITHGTDTLEETAFFLDATVNCGKPIVIVGAMRPSTTISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 6 - protein, 378 aa, Artificial sequence
  Mutated from Aspergillus niger
MPLKPILLSALASLASTSPLLYSRTTNETFVFTNANGLNFTQMNTTLPNVTIFATGGTIAGSGSSSTATTGYTSGAVGVLSLIDAVPSMLDVANVAGVQVANVGSEDITSDILISMSKNLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPCVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 7 - protein, 378 aa, Artificial sequence
  Mutated Aspergillus niger
MPLKPILLSALASLASASPLLYSRTTNETFVFTNANGLNFIQMNTTLPNVTIFATGGTIAGSDSSPTATTGYTSGAVGVLSLIDAVPVMLDVANVAGVQVANVGSEDITSDILISMSKKLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPAKPTGKVAFDITNVTEIPCVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 8 - protein, 378 aa, Artificial sequence
  Mutated from Aspergilus niger
MPLKPILLSALASLASASPLLYSRTTNETFVFTNANGLNFTQMNTTLPNVTIFATGGTIAGSHSSSTATTGYTSGTFGVLSLIDAVPSMLDVANVAGVQVVNVGSEDITSGILISMSKKLNRVVCEDPTMAGAVITHGTDTLEETAFFLDATVNCGKPIVLVGAMRPSTTISADGPFNLLEAVTVAASTSARDRGAMVVMNDRIASAYYVTKTNANTMDTFKAMEMGYLGEMISNTPFFFYPPAKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADMFALGTDA

SEQ ID NO: 9 - protein, 378 aa, Artificial sequence
  Mutated from Aspergillus niger
MPLKPILLSALASLAAASPLLYSRTTNETFVFTNANGLNFTQMNTTLPNVTIFATGGTIAGSGSSSTATTGYTSGTVGVLSLIDAVPSMLDVANVAGVQVANVGSEDITSDILISMSKNLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 10 - protein, 378 aa, Artificial sequence
  Mutated from Aspergillus niger
MPLKPILLSALASLASASPLLYSRTTNGTFVFANANGLNFTQMNTTLPNVTIFATGGTIAGSHSSSTATTGYTSGAVGVLSLIDAVPSMLDVANVAGVQVANVGSEDITSDILISMSKKLNRVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA

SEQ ID NO: 11 - DNA, 1137 bp, Artificial sequence
  Mutated from Aspergillus niger
atgcctctcaagccgattctcctgtctgccctggccagtctcgccgcggcctctccgctgctctactcgcggaccaccaatgaaaccttcgtcttcaccaatgccaatggcctcaacttcacccagatgaacaccaccctgccgaacgtgaccattttcgcaacgggtggtaccatcgcgggctccggttccagctcaaccgccacgactggctacacctccggagcagtcggggtcctgtccctcatcgatgcggtgccatccatgctggatgtggccaatgttgccggcgtccaggtggccaacgtgggaagcgaggatatcacctctgacatcctgatttccatgtccaagaagctgaaccgcgttgtatgtgaggacccgaccatggccagtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctattatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggttggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 12 - DNA, 1137 bp, Artificial sequence
  Mutated from Aspergillus niger
atgcctctcaagccgattctcctgtctgccctggccagtctcgcctcggcctctccgctgctctactcgcggaccaccaatgaaaccttcgtcttcaccaatgccaatggcctcaacttcatccagatgaacaccaccctgccgaacgtgaccattttcgcaacgggtggtaccatcgcgggctccggttccagctcaaccgccacgaccggctacacctccggagcagtcggggtcctgtccctcatcgatgcggtgccagtgatgctggatgtggccaatgttgccggcgtccaggtggccaacgtgggaagcgaggatatcacctctggcatcctgatttccatgtccaagaagctgaaccacgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 13 - DNA, 1137 bp, Artificial sequence
  Mutated from Aspergillus niger
atgcctctcaagccgattctcctgtctgccctggccagtctcgcctcggcctctccgctgctctactcgcggaccaccaatgaaaccttcgtcttcaccaatgccaatggcctcaacttcatccagatgaacaccaccctgccgaacgtgaccattttcgcaacgggtggtaccatcgcgggctccggttccagctcaaccgccacgaccggctacacctccggagcagtcggggtcctgtccctcatcgatgcggtgccattcatgctggatgtggccaatgttgccggcgtccaggtggccaacgtgggaagcgaggatatcacctctgacatcctgatttccatgtccaagaagctgaaccgcgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 14 - DNA, 1137 bp, Artificial sequence
  Mutated from Aspergillus niger
atgcctctcaagccgattctcctgtctgccctggccagtctcgcctcggcctctccgctgctctactcgcggaccaccaatgaaaccttcgtcttcaccaatgccaatggcctcaacttcacccagatgaacaccaccctgccgaacgtgaccattttcgcaacgggtggtaccatcgcgggctcccactccagctcaaccgccacgaccggctacacctccggaacatttggggtcctgtccctcatcgatgcggtgccatccatgctggatgtggccaatgttgccggcgtccaggtggtcaacgtgggaagcgaggatatcacctctgacatcctgatttccatgtccaagaagctgaaccgcgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacgaccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 15 - DNA, 1137 bp, Artificial sequence
  Mutated from Aspergillus niger
atgcctctcaagccgattctcctgtctgccctggccagtctcgcctcgacctctccgctgctctactcgcggaccaccaatgaaaccttcgtcttcaccaatgccaatggcctcaacttcacccagatgaacaccaccctgccgaacgtgaccattttcgcaacgggtggtaccatcgcgggctccggttccagctcaaccgccacgactggctacacctccggagcagtcggggtcctgtccctcatcgatgcggtgccatccatgctggatgtggccaatgttgccggcgtccaggtggccaacgtgggaagcgaggatatcacctctgacatcctgatttccatgtccaagaacctgaaccgcgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccctgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 16 - DNA, 1137 bp, Artificial sequence
  Mutated from Aspergillus niger
atgcctctcaagccgattctcctgtctgccctggccagtctcgcctcggcctctccgctgctctactcgcggaccaccaatgaaaccttcgtcttcaccaatgccaatggcctcaacttcatccagatgaacaccaccctgccgaacgtgaccattttcgcaacgggtggtaccatcgcgggctccgattccagcccaaccgccacgactggctacacctccggagcagtcggggtcctgtccctcatcgatgcggtgccagtaatgctggatgtggccaatgttgccggcgtccaggtggccaacgtgggaagcgaggatatcacctctgacatcctgatttccatgtccaagaagctgaaccgcgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctattatgtgaccaagaccaatgccaacactatggacaccttcaaggccatggagatgggctaccttggcgagatgatctccaacacccctttcttcttctacccgcccgccaagccaaccggtaaggtggcctttgacatcaccaacgtgactgagatcccctgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 17 - DNA, 1137 bp, Artificial sequence
  Mutated from Aspergillus niger
atgcctctcaagccgattctcctgtctgccctggccagtctcgcctcggcctctccgctgctctactcgcggaccaccaatgaaaccttcgtcttcaccaatgccaatggcctcaacttcacccagatgaacaccaccctgccgaacgtgaccattttcgcaacgggtggtaccatcgcgggctcccactccagctcaaccgccacgaccggctacacctccggaacatttggggtcctgtccctcatcgatgcggtgccatccatgctggatgtggccaatgttgccggcgtccaggtggtcaacgtgggaagcgaggatatcacctctggcatcctgatttccatgtccaagaagctgaaccgcgttgtatgtgaggacccgaccatggccggtgctgtcatcacccacggcaccgacaccctggaggagactgccttcttcctggacgccactgtcaactgtggcaagccaattgtcctcgtgggtgccatgcgcccatccacgaccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgccaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacatgtttgctctgggcacggatgcgtaa

SEQ ID NO: 18 - DNA, 1137 bp, Artificial sequence
  Mutated from Aspergillus niger
atgcctctcaagccgattctcctgtctgccctggccagtctcgccgcggcctctccgctgctctactcgcggaccaccaatgaaaccttcgtcttcaccaatgccaatggcctcaacttcacccagatgaacaccaccctgccgaacgtgaccattttcgcaacgggtggtaccatcgcgggctccggttccagctcaaccgccacgactggctacacctccggaacagttggggtcctgtccctcatcgatgcggtgccatccatgctggatgtggccaatgttgccggcgtccaggtggccaacgtgggaagcgaggatatcacctctgacatcctgatttccatgtccaagaacctgaaccgcgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 19 - DNA, 1137 bp, Artificial sequence
  Mutated from Aspergillus niger
atgcctctcaagccgattctcctgtctgccctggccagtctcgcctcggcctctccgctgctctactcgcggaccaccaatggaaccttcgttttcgccaatgccaatggcctcaacttcacccagatgaacaccaccctgccgaacgtgaccattttcgcaacgggtggtaccatcgcgggctcccattccagctcaaccgccacgactggctacacctccggagcagtcggggtcctgtccctcatcgatgcggtgccatccatgctggatgtggccaatgttgccggcgtccaggtggccaacgtgggaagcgaggatatcacctctgacatcctgatttccatgtccaagaagctgaaccgcgttgtatgtgaggacccgaccatggccggtgctgtcatcacccacggcaccgacaccctggaggagactgccttcttcctggacgccactgtcaactgtggcaagccaattgtcatcgtgggtgccatgcgcccatccacggccatctcagctgacgggcccttcaatctgctcgaagccgtgacggtggctgcctccacgtcggcgcgcgatcgcggtgccatggtggtcatgaacgatcgcattgcctcggcctactatgtgaccaagaccaatgccaacactatggacaccttcaaggccatggagatgggctaccttggcgagatgatctccaacacccctttcttcttctacccgcccgtcaagccaaccggtaaggtggcctttgacatcaccaacgtgactgagatcccccgtgtggacattctgttttcttatgaggacatgcacaacgacaccctctacaacgccatctccagtggtgcccagggaattgtgattgccggggctggtgctggaggcgtcacaacctccttcaatgaggctatcgaggatgtcatcaaccgtttggagatccctgtcgtgcagagtatgcgcacagtcaatggggaagtgccactgtcagacgtgagcagcgacaccgccacccacatcgccagtggatacctaaacccgcagaagtcccgcattctgttgggattgctgctatcccagggaaagaatatcaccgaaatcgctgacgtgtttgctctgggcacggatgcgtaa

SEQ ID NO: 20 - protein, 378 aa, Artificial sequence
  ASN 02 (Mutated from Aspergillus niger)
MPLKPILLSALASLASASPLLYSRTTNETFVFTNANGLNFTQMNTTLPNVTIFATGGTIAGSGSSSTATTGYTSGAVGVLSLIDAVPSMLDVANVAGVQVANVGSEDITSGILISMSKKLNHVVCEDPTMAGAVITHGTDTLEETAFFLDATVNCGKPIVIVGAMRPSTAISADGPFNLLEAVTVAASTSARDRGAMVVMNDRIASAYYVTKTNANTMDTFKAMEMGYLGEMISNTPFFFYPPVKPTGKVAFDITNVTEIPRVDILFSYEDMHNDTLYNAISSGAQGIVIAGAGAGGVTTSFNEAIEDVINRLEIPVVQSMRTVNGEVPLSDVSSDTATHIASGYLNPQKSRILLGLLLSQGKNITEIADVFALGTDA